(19) United States
(12) Patent Application Publication
(10) Pub. No.: US 2010/0089937 A1
(43) Pub. Date: Apr. 15, 2010

(54) CHILD PROOF MEDICATION PACKAGING SYSTEM AND METHOD

(76) Inventors: Lawrence Luciano, Somerville, NJ (US); Leon A. Marucchi, Annandale, NJ (US)

Correspondence Address: KERR IP GROUP, LLC;MICHAEL A. KERR, 59 DAMONTE RANCH PARKWAY SUITE B, BOX 370, RENO, NV, 89521, US

(21) Appl. No.: 12/418,436

(22) Filed: Apr. 3, 2009

Related U.S. Application Data
(63) Continuation-in-part of application No. 11/923,321, filed on Oct. 24, 2007.
Application No. 11/923,321 is a continuation-in-part of application No. 11/796,123, filed on Apr. 25, 2007.
Application No. 11/796,123 is a continuation-in-part of application No. 11/796,124, filed on Apr. 25, 2007.
Application No. 11/796,124 is a continuation-in-part of application No. 11/796,125, filed on Apr. 25, 2007.
Application No. 11/796,125 is a continuation-in-part of application No. 11/241,783, filed on Sep. 30, 2005.
(60) Provisional application No. 61/042,263, filed on Apr. 3, 2008.
(60) Provisional application No. 61/042,262, filed on Apr. 3, 2008.
(60) Provisional application No. 60/795,370, filed on Apr. 26, 2006.
(60) Provisional application No. 60/795,446, filed on Apr. 26, 2006.
(60) Provisional application No. 60/795,413, filed on Apr. 26, 2006.
(60) Provisional application No. 60/845,341, filed on Sep. 18, 2006.
(60) Provisional application No. 60/615,267, filed on Oct. 1, 2004.

(51) Int. Cl. B65D 83/04; B65D 85/00

(52) U.S. Cl. 221/1; 221/133

(57) ABSTRACT

A child proof medication packaging system and method is described. The child proof medication packaging system includes a dispenser and a child safety component. The dispenser includes a plurality of medicament containers housing a plurality of different medications and an aperture that allows the medicament containers to exit the dispenser through the aperture. The child safety component associated with the aperture in the dispenser is adjustable to a first position and a second position. The first position prevents the medicament containers from passing through the aperture and the second position permits the medicament containers to pass through the aperture.

### CROSS REFERENCE TO RELATED APPLICATIONS

This application Ser. No. 12/418,436 is related to co-filed patent application Ser. Nos. 12/418,418 and 12/418,422, both having a filing date of Apr. 3, 2009. The present patent application claims priority from provisional patent applications 61/045,160, 61/045,166, and 61/045,171, all filed Apr. 15, 2008. The present application is a continuation-in-part of nonprovisional application Ser. No. 11/923,321, filed Oct. 24, 2007, which is a continuation-in-part of nonprovisional patent application Ser. Nos. 11/796,123, 11/796,124, and 11/796,125 filed Apr. 25, 2007, which all claim benefit of provisional applications 60/795,370, 60/795,446, and 60/795,413, filed Apr. 26, 2006, and provisional application 60/854,341, filed Oct. 24, 2006. This application is a continuation-in-part of nonprovisional application Ser. No. 11/241,783, which is related to provisional application 60/615,267, filed Oct. 1, 2004. All applications listed in this paragraph are hereby incorporated by reference.

### FIELD OF THE INVENTION

This invention relates to a spiral medication packaging system and method. More particularly, the spiral medication packaging system and method provides a spiral packaging solution for a plurality of different tablets that are housed by a plurality of medicament containers.

### BACKGROUND

Approximately fifty to seventy percent of all prescription medications in the U.S. are taken incorrectly. The effects of this prescription misuse account for 3.1 million nursing home admissions each year. More patients in the U.S. die each year from medication mismanagement than from AIDS and automobile accidents combined, and it is estimated that 125,000 deaths per year are caused by improper use of prescription drugs.

One of the major problems in taking prescribed daily medications emanates from patients having to take more than one medication in the form of pills or tablets. A principal concern is determining whether all medications are in compliance with the prescribed daily regimen. Many times this concern is compounded by the requirement that portions of the different medications must be taken at different times during the day.

The fear of taking improper dosages of prescribed medication can be particularly acute in the elderly, many of whom have some degree of mental dementia and can easily be confused as to whether they have taken all of their medications at the correct time. Some patients have difficulty sorting out the medications prior to taking them and taking the medication in a timely manner. Providing medications to disabled or incapacitated individuals can also be complicated because one caregiver may oversee the medication of many patients.

One solution to the problem of taking multiple medications is to pre-package the multiple medications so that users can take the pre-packaged medications at a predetermined time. Generally, these methods of pre-packaging medications are targeted to patients that may lack maturity or mental capacity to take the correct medications at the correct time. For example, young children in a school or campground and elderly individuals in elder care centers or nursing homes are target groups for the pre-packaging of medications. Some of the pre-packaged medications are placed in a small plastic bag, which may be easily misplaced. Other pre-packaged medications are placed in sealed cups that are difficult to open.

Solid dose medicines are typically sold in vials, with each specific medicine type and dosage in a separate vial. When multiple medications are required to be taken at the same time (dosage period), typically, a patient will be required to extract the proper medicine from each vial. After the medicine is taken from the vial, there is no way to easily determine that it was actually taken (unless a pre and post count is done). Also, typically the patient (or caregiver) is responsible for determining the proper medicine to take at the proper time. This can be particularly confusing when medicines or medicine groups need to be administered on an irregular schedule (i.e. once a week, every other day, etc.).

There are compartmentalized sealed cups available commercially to assist patients and/or health care providers with this process. These, however, require the patient or caregiver to presort all medicines and to load them into their proper dosage period compartment, typically on weekly basis. This is time consuming and subject to human error. Additionally, these sealed cups lack indicia for identifying individual medications within each cup or pouch, which raises the likelihood of administration error.

There are also some products that are commercially available or are available through institutional groups or hospitals that contain the presorted medicines in individual pouches. The individual pouches can be pre-labeled/printed with the proper time and date. These pouches are filled using automatic equipment that is subject to machine error and possible medicine breakage. Additionally, these pre-filled pouches lack enough printed information to enable a caregiver or patient to determine at a glance which medicament is which. While the patient or caregiver may be able to see the medicaments (through the transparent pouch) and to read their names (printed on the pouches), the patient or caregiver has no way to know which medicament matches to which drug name/dosage amount without consulting other literature.

### SUMMARY

A child proof medication packaging system and method is described. The child proof medication packaging system includes a dispenser and a child safety component. The dispenser includes a plurality of medicament containers housing a plurality of different medications and an aperture that allows the medicament containers to exit the dispenser through the aperture. The child safety component associated with the aperture in the dispenser is adjustable to a first position and a second position. The first position prevents the medicament containers from passing through the aperture and the second position permits the medicament containers to pass through the aperture.

Another illustrative child proof medication packaging system includes the dispenser and a security means for preventing access to the medicament containers in the dispenser. The security means prevents the medicament containers from passing through the aperture when in the first position and permits the medicament containers to pass through the aperture when in the second position.

The method for dispensing medication includes housing a plurality of medicament containers in a dispenser, in which each medicament container includes medication associated with a dosage regimen. The dispenser includes an aperture that allows the medicament containers to exit the dispenser through the aperture and includes a child safety component associated with the aperture that is adjustable to a first position and to a second position. The method then proceeds to prevent the medicament containers from passing through the aperture when the child safety component is in a first position. The method permits the medicament containers to pass through the aperture when in the second position.

Additionally, one of the illustrative embodiments includes adjusting the child safety component to the second position and then proceeding to advance the medicament containers towards the aperture such that one of the medicament containers passes through the aperture. The method then proceeds to return the child safety component to the first position.

### DRAWINGS

The present invention will be more fully understood by reference to the following drawings which are for illustrative, not limiting, purposes.

FIG. 1 is a perspective view of one embodiment of a spiral packaging system.

FIG. 2 is an exploded view of the spiral packaging system of FIG. 1.

FIG. 3 is a perspective view of the spiral packaging system of FIGS. 1 and 2 shown without the second shell portion and showing a plurality of containers.

FIG. 4 is a perspective view of one embodiment of a spiral packaging system shown with a medicament container.

FIG. 5 is another perspective view of the spiral packaging system of FIG. 4 shown without the medicament container.

FIG. 6 is a perspective view of a housing portion of the spiral packaging system of FIG. 5.

FIG. 7 is a perspective view of the spiral packaging system of FIG. 1 through FIG. 3 shown with a compliance wheel to provide a patient medication management system.

FIG. 8 is an exploded view of the patient medication management system of FIG. 7 shown with the compliance wheel removed.

FIG. 9 is a perspective view of another embodiment of a patient medication management system.

FIG. 10 is a perspective view of the patient medication management system of FIG. 9 shown with the compliance wheel removed and with the internal shell components omitted for clarity.

FIG. 11 shows the second side of the housing of the patient medication management system of FIG. 10.

FIG. 12 is a perspective view of a portion of the patient medication management system of FIG. 9 showing a portion of a compliance wheel.

FIGS. 13A, 13B and 13C are perspective views of a compliance wheel.

FIGS. 14A and 14B are perspective views of one embodiment of a medicament container.

FIG. 15 is a perspective view of a compliance wheel interacting with a medicament container.

FIG. 16 is a perspective view of one embodiment of a patient medication management system shown with a portion of the housing removed to illustrate interaction of a compliance wheel with a plurality of medicament containers.

FIG. 17 is another perspective view of the spiral packaging system of FIG. 17 in a standing position.

FIG. 18 is another perspective view of the spiral packaging system of FIG. 17.

FIG. 19A presents a portion of a label strip, usable with the patient medication management system, that includes patient name and barcode.

FIG. 19B shows a portion of a label strip including part of the precaution information key for each drug in the containers.

FIG. 19C shows a portion of a label strip that includes part of the drug information section.

FIG. 19D shows a portion of a label strip that includes part of the drug information section.

FIG. 19E shows a portion of a label strip that includes the patient name and time period for administration.

FIG. 20 is a perspective view of an embodiment of a spiral packaging system with a transparent exterior, shown with a compliance wheel and a plurality of medicament containers.

FIG. 21 is a perspective view of two spiral packaging systems shown with child safety elements to provide a child proof medication management system.

FIG. 22 is a perspective view of a portion of the child proof medication management system of FIG. 21.

FIG. 23 is a front elevation view of the portion of the child proof medication management systems of FIG. 21 showing operation of a child safety component of the spiral packaging system.

FIG. 24 is a perspective view of a portion of another embodiment of a child proof medication management system.

FIG. 25A through FIG. 25F are side elevation views showing a patient medication management system mounted on a stand in variety of different orientations.

### DESCRIPTION

Persons of ordinary skill in the art will realize that the following description is illustrative and not in any way limiting. Other embodiments of the claimed subject matter will readily suggest themselves to such skilled persons having the benefit of this disclosure. It shall be appreciated by those of ordinary skill in the art that the systems and apparatus described hereinafter may vary as to configuration and as to details. Additionally, the methods may vary as to details, order of the actions, or other variations without departing from the illustrative methods disclosed herein.

The systems, apparatus and methods described herein provide correct medications and dosages at the appropriate times. The spiral packaging is designed with clear images and names to simplify the process of determining the appropriate medications to take at the appropriate time. Additionally, the patient medication management system and method described herein eliminates the worry of taking the wrong medication at the wrong time, and eliminates the need to manually open and organize prescriptions each week. Furthermore, the system and method described herein gathers all the patient's medicines together in one package and clearly shows the proper dosage time.

The systems, apparatus and methods described herein provide a compliant packaging solution. Compliance packaging generally includes three aspects: firstly, an action is initiated by a patient and/or caregiver with the compliance package; secondly, the compliance package dispenses at least one tablet as a result of the action taken by the patient and/or caregiver; and thirdly, the compliance package records the dispensing of the tablet. One illustrative example of a “compliance package” is the birth control “dial pack” package, in which there are twenty-eight tablets in a blister package that are in a circular configuration (not shown). To consume the tablet, the patient pushes on the transparent plastic material and the tablet pierces a foil backing. After the tablet is dispensed from the dial package, a record is left on the dial pack package, i.e. a pressed plastic housing and pierced foil backing.

The systems, apparatus and methods described herein satisfy the requirements for a compliance package because, firstly, an action is required by the patient or caregiver that requires identifying the appropriate dosage period, e.g. morning, and selecting the appropriate pouch or container. Secondly, the patient opens the appropriate container and consumes the medication. Thirdly, the patient or caregiver records the consumption of the medication by removing the container, which advances the compliance wheel of the packaging system as described in further detail below.

The systems, apparatus and methods described herein provide assurances of the proper dosages at the proper period. Additionally, caregivers and patients get the assurance that the patient is getting the right medications and staying compliant with those medications. Furthermore, a time saving solution for dispensing tablets, medications and vitamins is described.

The systems, apparatus and methods described herein also provide for a child resistant medication management system that allows all of the features of a compliant medication management system together with features to prevent improper use by children.

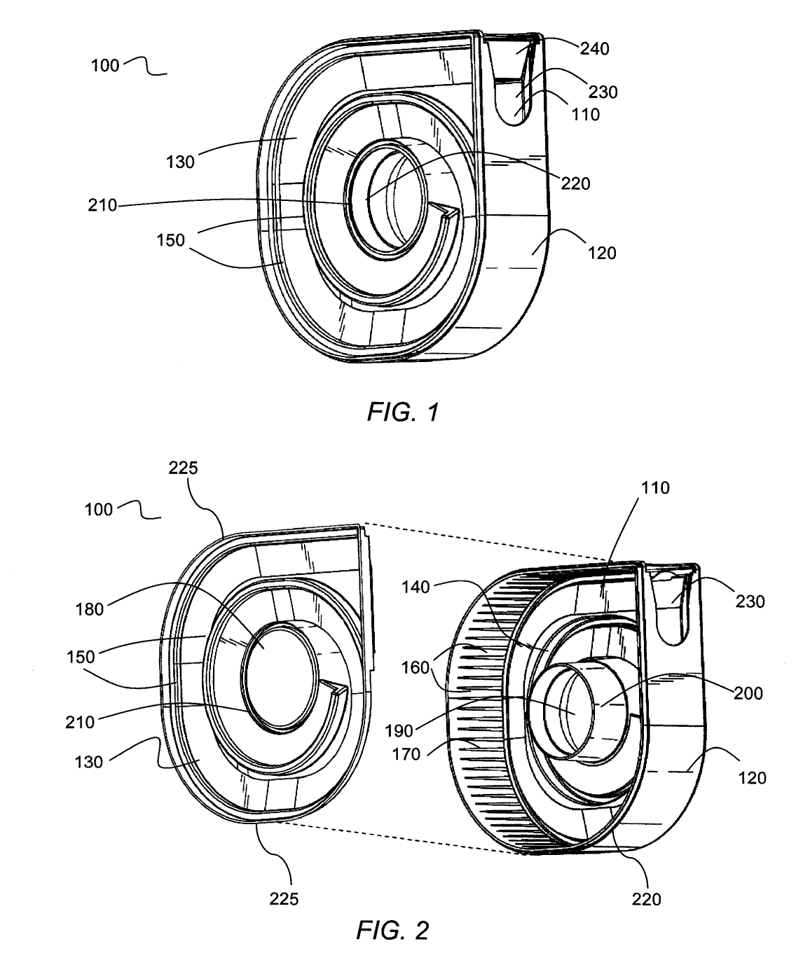

Referring now to FIGS. 1 through 3, there is shown one embodiment of a spiral packaging medication dispenser or system 100. The spiral packaging system 100 comprises first and second opposing shell portions 110, 130, and an outer wall 120. In the embodiment of FIG. 1, shell portions 110 and 130 are configured to fit together with outer wall 120 circumferentially positioned about shell portions 110 and 130 to define or form a medication dispenser.

The first 110 and second 130 shell portions include spiral tracks 140 and 150, respectively. Tracks 140 and 150 are configured to hold a plurality of medication receptacles or containers (not shown). Grooves or ridges 160 on the interior surface 170 of wall 120 aid in positioning the pill receptacles within the spiral packaging system 100 and advancing the receptacles along the tracks 140 and 150 as described further below.

The first 110 and second 130 shell portions include central openings 180 and 190, respectively. First shell central opening 180 and second shell opening 190 both include a circular edge or flange, denoted as 200 and 210, respectively. Flanges 200 and 210 are structured and configured to engage each other and secure the first 110 and second 130 shell portions together. In the embodiment of FIGS. 1 and 2, flange 200 is of slightly larger diameter than flange 210 such that flange 210 fits within flange 200 to hold shell portions 110 and 130 together. Flanges 200 and 210 may be tapered as shown in FIGS. 1 and 2 to allow flanges 200 and 210 to engage each other by tension, and to provide the end user with an ergonomically friendly packaging system. Outer wall 120 includes a circumferential flange 220 that engages a corresponding lip 225 on second shell portion 130. Flange 220 and lip 225 interfit and engage each other to hold second shell portion 130 to wall 120. The central openings 180 and 190 in shell components 110 and 130 allow the apparatus 100 to be rotatably mounted on bracket or other element as described further below.

The outer wall 120 of spiral packaging system 100 further comprises an opening or dispensing aperture 230 configured to allow medication/pill receptacles or containers (not shown) to be removed or ejected from the spiral packaging system 100. The container aperture or opening 230 is positioned on the shell wall portion 120 of the apparatus and is of sufficient size to allow medicament containers to pass through sequentially or one at a time as described further below.

First shell 110, second shell 130 and wall portion 120 of the spiral medication packaging system 100 may be manufactured from molded plastics, composite materials, engineering resins, metals or metal alloys and the like, or combinations thereof. The various components of the apparatus 100 may be made of transparent, semi-transparent or opaque materials that aid the patient or caregiver in determining the number of pill receptacles left in the spiral packaging system 100. First shell 110, second shell 130, and shell wall 120 may be manufactured separately and configured to enable the first 110 and second 130 shell portions to snap fit together or may be configured to be joined together using adhesives or other fastening means. In certain embodiments, either of the first 110 or second 130 shell portion and the shell wall 120 section may be manufactured as integral components of a single work piece (e.g. injection molded) with the remaining shell portion configured to attach thereto to form the spiral packaging system 100 by snap fitting, tensional engagement or other fastening means.

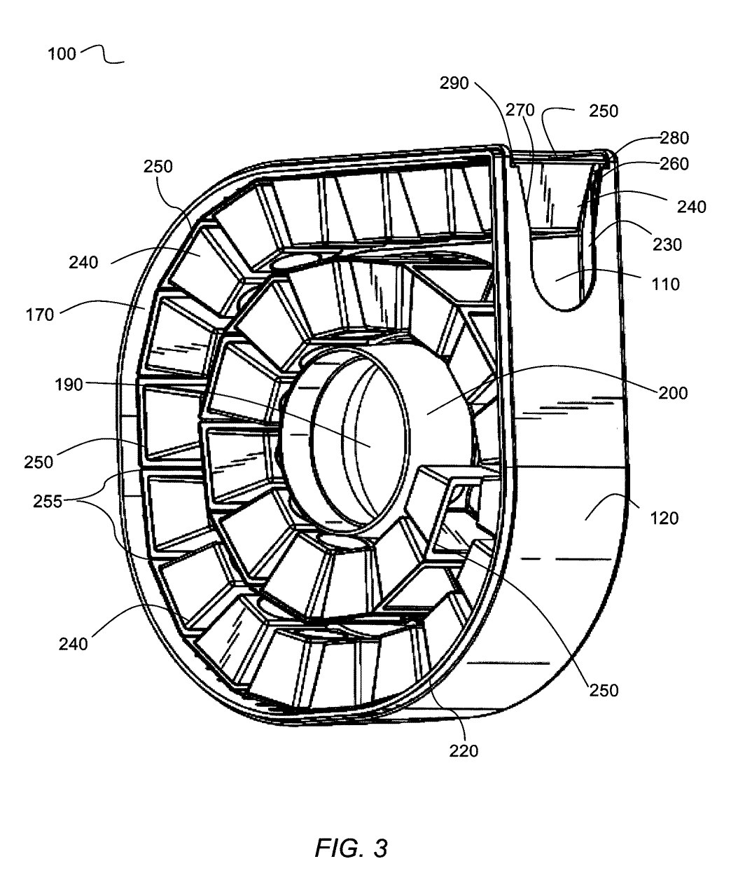

Referring also to FIG. 3, there is shown a perspective of the spiral packaging system 100 of FIGS. 1 and 2 with shell portion 130 removed to show a plurality of tablets or medicament containers 240 configured to hold tablets, caplets, pills, and other such medicaments. The containers 240 in this embodiment each comprise a flanged top edge 250 configured to fit into and interact with the interior spiral tracks 140 and 150 respectively of the first 110 and second 130 shell portions of the apparatus 100. The tubular flange 200 of the first shell central opening 190 aids in the alignment of the plurality of containers 240 within spiral tracks 140 and 150. The top edge flanges 250 of adjacent containers 240 may be joined together by a frangible interface or connector 255 that is detached or broken by a user. Medicament containers 240 may be made, for example, of molded plastic or metal foil or a combination thereof. Medicament containers 240 may also include a frangible or removable top portion (not shown) as described below that may be penetrated or removed by user to access medication within containers 240.

The apparatus 100 in many embodiments is configured to hold approximately one month's supply that is generally a 30-day supply, but may also include about 28 to about 31 containers of a patient's medication. In some embodiments the spiral packaging system 100 may be configured to only hold one week of medication when the patient needs medication more than once a day. For example, when a patient will require three different medication dosages during a single day, the spiral packaging system 100 may be configured to support 21 containers 240 (i.e. 3 doses per day×7 days=21 containers 240). Various other numbers of containers 240 may be used depending upon the particular use.

Container opening 230 in wall section 120 further comprises first and second edges 260 and 270 that are configured to the shape of the containers 240 to allow the containers to pass through opening 230. First 260 and second 270 edges include top indentations 280 and 290 respectively that are configured to allow the flanged edge 250 of the container 240 to pass from the interior spiral groove(s) through the container opening 230 and out of the spiral packaging system 100. The container opening 230 in many embodiments is configured to include a childproof component (not shown) as is described further below.

In certain embodiments first 110 and second 130 shell portions are reversibly attachable and detachable to allow a user to open the apparatus 100 by detaching shell portion 110 from shell portion 130 so that medicament containers 240 may be inserted into the apparatus 100, after which the shell portions are re-attached. The apparatus 100 is thus re-usable as the user can replace the medicament containers 240 therein when used up. In other embodiments the medicament containers 240 may be inserted into the apparatus 100 by a health care provider or pharmaceutical supplier, with shell portions 110 and 130 then being permanently attached or adhered together so that the end user or patient cannot open the apparatus 100 or access the medicament containers 240 except via the dispensing aperture 230.

To use the apparatus 100, medicament containers 240 are arranged on track 140 or 150 in a spiral arrangement to conform to the shape of tracks 140 and 150, with flanges 250 of containers fitting over the outer edge of tracks 140 and 150. In many embodiments medicament containers 240 are arranged in a linear chain with each medicament container 240 joined to adjacent containers 240 by the frangible interface 255. The medicament containers 240 are positioned so that the first container 240 is adjacent opening 230 as shown in FIG. 3. Shell portions 110 and 130 are then attached to each other, leaving the medicament containers arranged along tracks 140 and 150. As the patient or user accesses the first medicament container 240 through dispensing aperture 230, the user pulls the first container 240 through aperture 230, with flanges 250 on containers 240 sliding along tracks 140 and 150 and over indents 280 and 290 of aperture 230. Opening 230 as shown is configured to allow a user to insert a finger through opening 230 to facilitate manipulation of container 240. As the first container 240 is withdrawn from aperture 230, the remaining containers 240 are advanced along tracks 140 and 150 towards aperture 230. The grooves or ridges 160 on the interior surface 170 of wall 120 provide some friction to containers 240 so that containers 240 move at a convenient rate along tracks 140 and 150. When the first or end-most container 240 has exited the apparatus 100 through aperture 230, the user detaches the first container 240 by breaking frangible interface 255. The next container 240 is positioned adjacent aperture 230 and may be accessed in the same manner. When the last medication container 240 has been withdrawn from the apparatus 100, the apparatus may be disposed of or opened to insert new medicament containers 240 and used again.

Each medicament container 240 includes, for example, the required medication for a particular time period. For each such time period, the patient accesses the container 240 adjacent to opening 230, and as each container is advanced and detached, the next container 240, having therein the medication for the next time period, is advanced to opening 230 for use in the following time period. The apparatus may include a timing element and alarm element (not shown), such as an oscillating quartz crystal timing device and a sound chip interfaced thereto, with the timing device and alarm chip powered by a small battery. When the proper time for medication has arrived according to the timing device, the alarm device may make a beeping or other alarm noise to alert the patient.

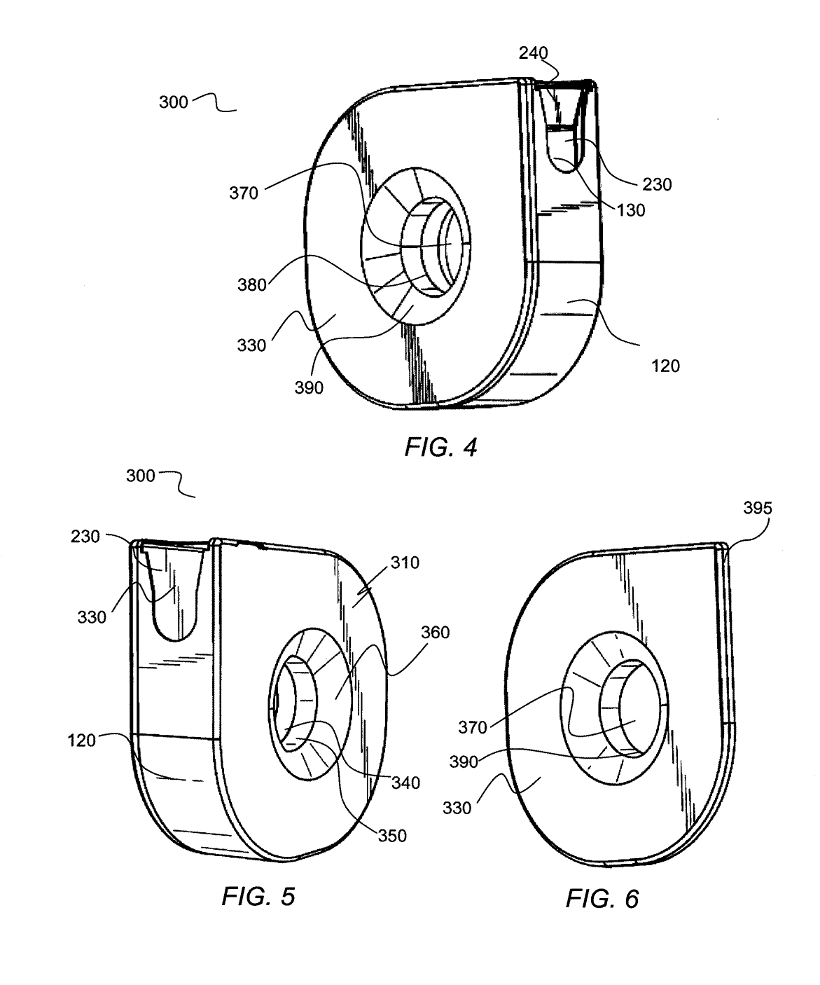

Referring now to FIGS. 4 through 6, there is shown another embodiment of a spiral medication dispenser or packaging system 300 in accordance with the invention, wherein like reference numbers are used to denote like parts. In the apparatus 300, first 110 and second 130 shell portions each include a corresponding first 310 and second 330 opposing facing or housing element. Housing element 310 includes a central opening 340, a circular flange 350 surrounding opening 340, and a tapered region 360 surrounding flange 350. Housing element 330 similarly includes a central opening 370, a flange 380 surrounding the opening 370, and a tapered region 390 surrounding flange 380.

Housing elements 310 and 330 are structured and configured to fit over first 110 and second 130 shell portions (FIGS. 1-3) respectively. In this regard, flanges 350 and 380 are designed to fit over flanges 200 and 210 of shell portions 110 and 130, and tapered portions 360 and 390 accommodate tracks 140 and 150 of shell portions 110 and 130 respectively. Housing elements 310 and 330 each include an outer flange 395 that is structured and configured to engage or otherwise interface with the outer lip 225 on shell portions 110 and 130. Housing elements 310 and 330 provide flat, smooth, easily cleanable outer surfaces to the apparatus 300 that allows facile handling and is pleasing to users. Housing elements 310 and 330 facilitate inclusion of compliance and/or child proofing elements as described further below. In other respects the apparatus 300 operates and is generally used in the same manner as described above for the apparatus 100.

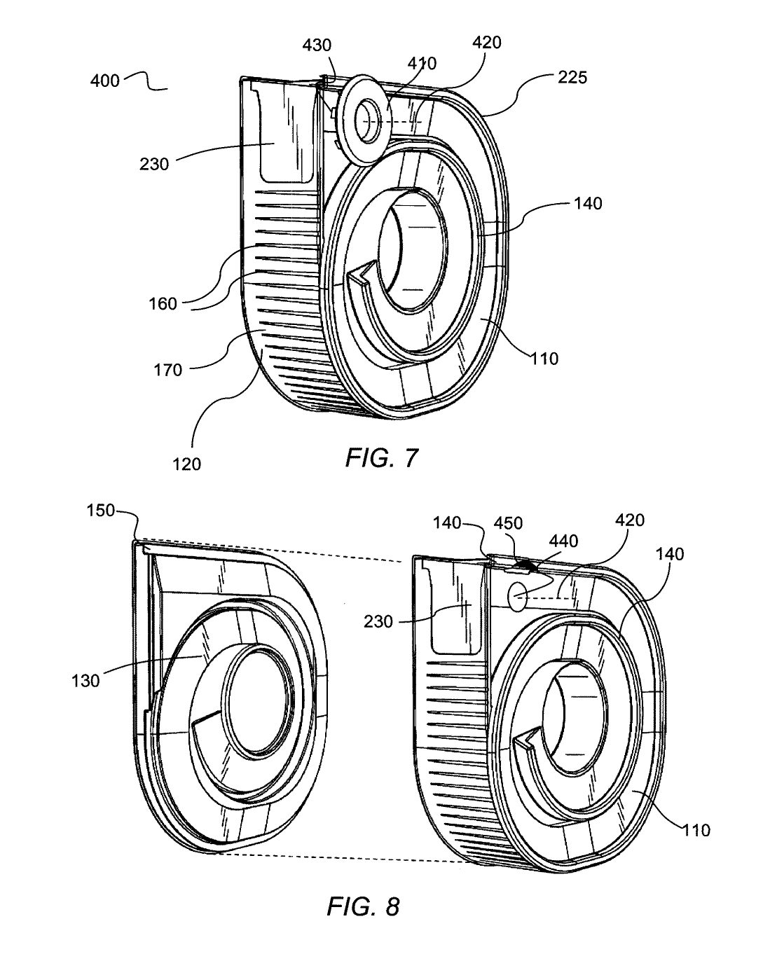

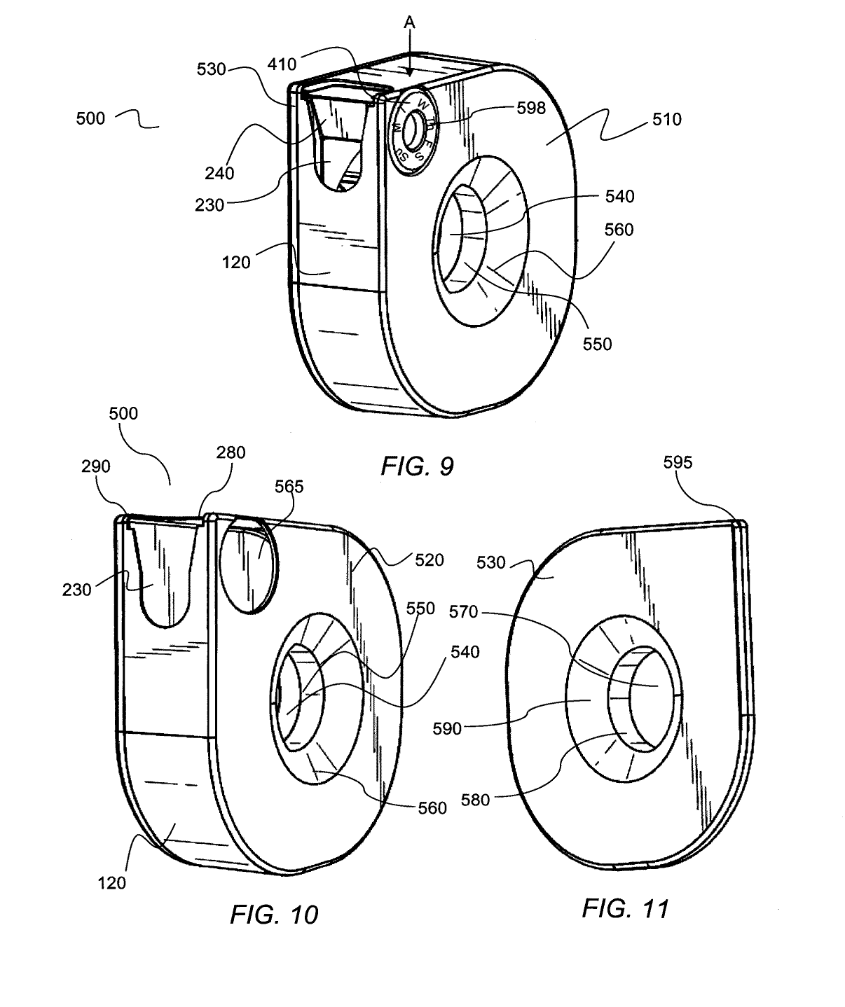

Referring now to FIG. 7, there is shown yet another embodiment of a spiral medication packaging system 400, wherein like reference numbers denote like parts. The spiral packaging system 400 shown in FIG. 7 comprises a compliance or drive wheel or sprocket 410 rotatably mounted on the first shell portion 110 of the apparatus 400 such that the compliance wheel 410 can rotate about a center axis 420. The wheel 410 includes a plurality of teeth 430 which are configured to interact with or engage containers 240 (not shown in FIG. 7 or FIG. 8) within spiral packaging system 400 when the containers 240 are adjacent or proximate to the wheel 410.

FIG. 8 is an exploded view of the spiral packaging system 400 of FIG. 7 shown with the compliance wheel 410 omitted for clarity. An opening 440 in the first shell portion 110 is structured and configured to receive and support compliance wheel 410 in a rotatable manner. A ratchet slot or opening 450 is configured to allow the teeth 430 of compliance wheel 410 access to the interior of the spiral packaging system 400 thus allowing the compliance wheel teeth 430 to interact with pill containers (not shown) when the apparatus 400 is filled with a chain of containers. The ratchet opening 450 is located proximate to the end portion of spiral track 140 of first shell portion 110, and proximate to container opening 230. The user may then detach and remove the container 240 that passes through opening 230 to access the medication contained therein.

In operation of the apparatus 400, a user manually rotates compliance wheel 410. The teeth 430 of the wheel 410 fit through slot 450 and engage the container 240 (FIG. 9) adjacent to slot 450, such that interaction of teeth 430 with container 240 during rotation of wheel 410 advance container 240 towards opening 230. After container 240 passes through opening 230, the user may detach the container 240 and access the medication therein as described above. Further rotation of wheel advances the next container 240 towards opening 230.

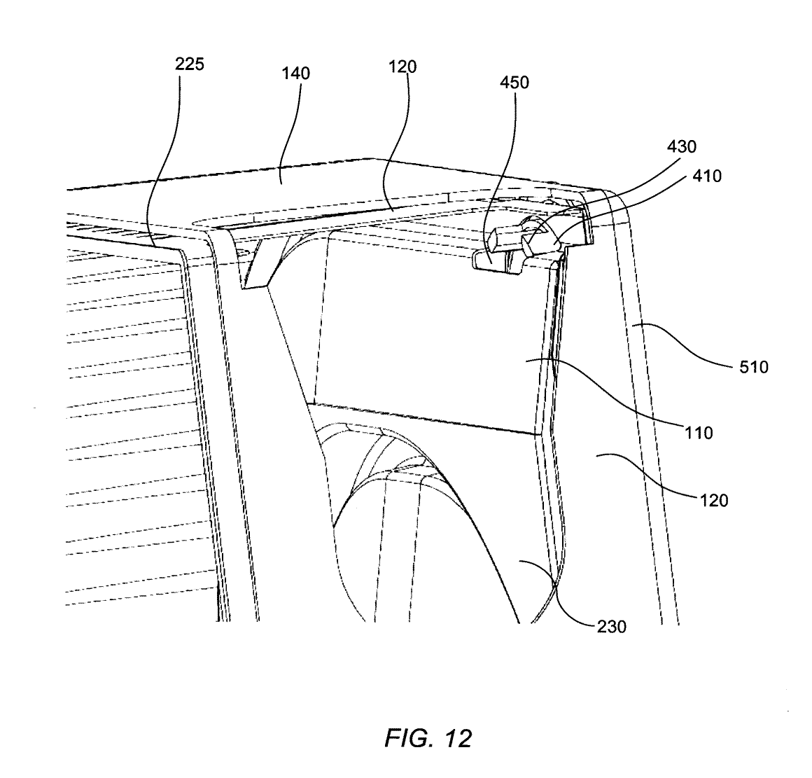

Referring now to FIGS. 9 through 12, there is shown another embodiment of a medication packaging system 500 wherein like reference numbers denote like parts. The apparatus 500 includes first and second opposing facings or housing elements 510 and 530. Housing element 510 includes a central opening 540, a circular flange 550 surrounding opening 540, and a tapered region 560 surrounding flange 550. Housing element 510 also includes an opening 565 structured and configured to rotatably accommodate compliance wheel 410. Housing element 530 includes a central opening 570, a flange 580 surrounding the opening 570, and a tapered region 590 surrounding flange 580. FIG. 12 shows sprocket teeth 430 of compliance wheel 410 extending through slot 450, as viewed through opening 230 in the apparatus 500.

Housing elements 510 and 530 are structured and configured to fit over first and second shell portions 110 and 130 (FIGS. 7-8) respectively. Flanges 550 and 580 thus are designed to fit over flanges 200 and 210 of shell portions 110 and 130, and tapered portions 560 and 590 accommodate tracks 140 and 150 of shell portions 110 and 130 respectively. Housing elements 510 and 530 each include an outer flange 595 (FIG. 11) that is structured and configured to engage or otherwise interface with the outer lip 225 (FIGS. 7 and 8) on shell portions 110 and 130. As in the embodiment of FIGS. 4-6, housing elements 510 and 530 provide flat, smooth, easily cleanable outer surfaces to the apparatus 300 that allows facile handling and is pleasing to users.

The compliance wheel 410 in FIG. 9 includes medical compliance indicia or indicators 598 associated with medicament containers 240, with indicia 598 configured to verify the location of medicament containers with respect to aperture 230 and whether or not a medicament container has already exited aperture 230 and been removed from the apparatus. Indicia 598 in this embodiment are temporal indicia and are shown as letters denoting days of the week, i.e., M for Monday, T for Tuesday, W for Wednesday etc., which are located circumferentially around wheel 410. The position of each indicium 598 on wheel 410 corresponds to the location of an associated or corresponding medicament container 240 within the apparatus 500 and provides an indication or verification for the user as to which medication container will next be dispensed through opening 230, and whether or not the medication container for a previous day or time period has already been removed from the apparatus 500.

Thus, for example, when wheel 410 is rotated such that the letter “W” is adjacent to arrow A, the user will know that the container 240 positioned in or adjacent to opening 230 includes the medication for Wednesday. The user further will know that the corresponding medication container 240 for Tuesday (represented by “T”) has already been removed from the apparatus 500, and that the container 240 containing Thursday (“Th”) medication remains in the apparatus 500 behind the container 240 having the Wednesday (“W”) medication. In this manner, the user can confirm or verify whether or not the appropriate medication for each particular day or other time period has been taken. In the embodiment of FIG. 9, counterclock-wise rotation of wheel 410 advances containers 240 towards opening. In the event that a user has advanced containers 240 too far, clock-wise rotation of wheel 410 may retract or move the containers back into the apparatus 500.

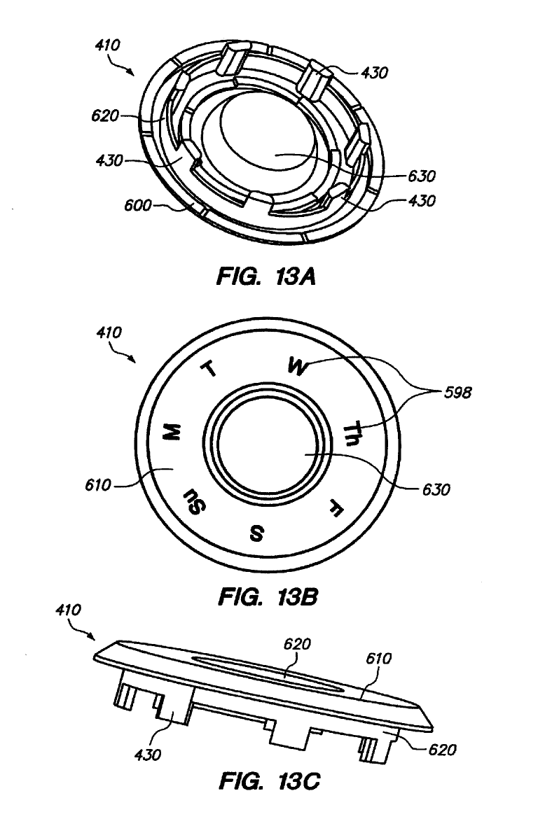

Referring to FIGS. 13A, 13B and 13C there is shown one embodiment of a compliance or drive wheel 410 in accordance with the invention. FIG. 13A shows a first or inner side 600 of a compliance wheel 410 comprising a plurality of sprocket teeth 430 configured to interact with containers held within a medication dispensing system. The number of teeth 430 elements on the compliance wheel 410 may be varied according to the type of medical compliance needed and the configuration of medicament containers. If, for example, the compliance is to be a 7-day cycle, the compliance wheel 410 comprises seven sprocket teeth 430, one for each day of the week. The teeth 430 are spaced and configured to interact with one medicament container at time.

FIG. 13B shows a second or outer side 610 of the compliance wheel 410 which includes indicia 598 representing each day of the week, i.e. M, T, W, Th, F, S and Su. The indicia corresponding to the days of the week are aligned and configured with the sprocket teeth 430 on the compliance wheel 410 so that the user can determine if the medication for a particular day has been given, as described above. The indicia 598 on compliance wheel 410 may comprise various time indicia other than, or in addition to, the days of the week as shown. For example, indicia 598 may include “AM” and “PM” indicia, or “morning”, “afternoon” and “evening” indicia, or “B”, “L” and “D” for “breakfast”, “lunch” and “dinner”, depending on the particular dosing regimen needed for the patient user of the apparatus 500. The number and positioning of sprocket teeth 430 on wheel 410 may be varied as required to accommodate different dosing requirements. The compliance wheel of the invention thus can provide for medical compliance even with complex patient dosage regimens.

The compliance wheel 410 is shown in FIGS. 13A-13C as being structured and configured for use with the apparatus 500 of FIGS. 9-11. Wheel 410 thus comprises a circular flange 620 configured to fit within the compliance wheel opening 565 of the first side 510 of the housing of the spiral packaging system 500 shown in FIG. 9. Sprocket teeth 430 are positioned on flange 610. Wheel 410 further comprises a central indented portion 630 configured to facilitate manual rotation of wheel 410 by a user.

While the compliance wheel 410 is shown as being configured for manual operation, various other mechanisms for turning or advancing compliance wheel 410 may be used. For example, a small battery-driven electric motor or a spring-actuated mechanism operating according to force applied to a button may be used to rotate wheel 410.

The compliance wheel 410 may be used with various medical packaging systems other then the spiral packaging system disclosed herein. Any pill packaging system comprising a plurality of containers 240 arranged in a chain can utilize a compliance wheel to aid the user in determining when the last dosage was given and when the next dosage is needed, as well as to move or advance medication containers within a dispensing device. For example, a rectangular box comprising seven medicament containers 240, i.e. a weekly dose, could use the compliance wheel to determine if the current day's dosage had been given. The medicament containers 240 could be arranged in a linear, circular or snake-like alternating chain configuration. Use of the compliance wheel in accordance with this disclosure provides for a variety of medication packaging systems with a dosage compliance feature.

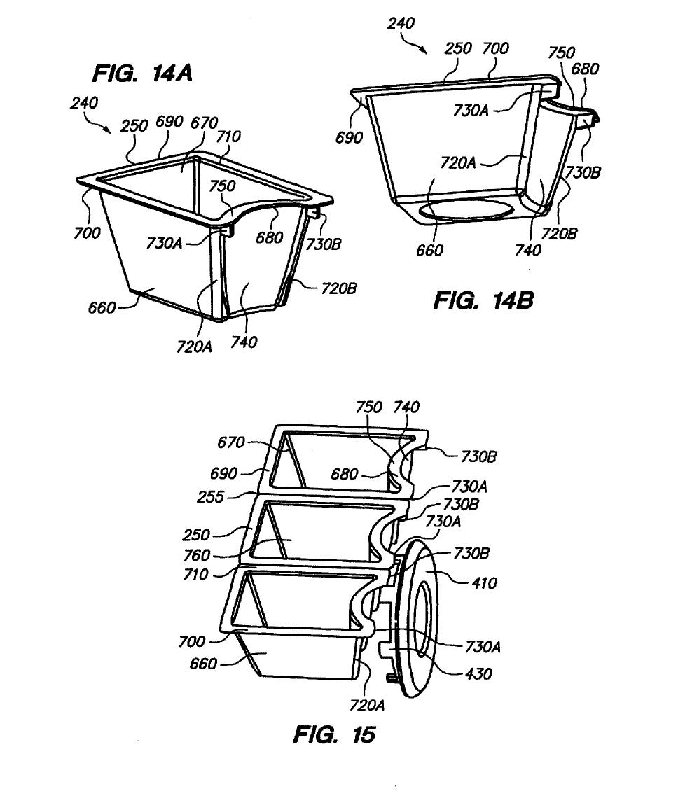

FIG. 14A, FIG. 14B and FIG. 15 show one embodiment of a medicament container or cup 240 for holding medication in the form of tablets, caplets, pills, capsules, powders liquids, gels, suppositories or other forms of medication. The medication within containers 240 may be prescription medication, vitamins, supplements, herbal formulations, or combinations thereof, intended to be ingested by or administered to a patient to improve the patient's health or well being. The medicament container 240 comprises a tapered body 660 with a cavity 670 for holding a plurality of pills or other medication. The container 240 includes a flanged top surface or edge 250 configured to be sealed with a lid (not shown). Container 240 may also include a frangible or removable top or lid (not shown) adjacent to flange 250. Breaking or removing the top allows a user to access the medication therein. The top may be transparent to allow a user to see the contents of container 240.

Generally, the flanged top edge 250 of container 240 comprises a first side 680 and second 690 side which are configured such that flange 250 interacts with the spiral grooves or tracks 140 and 150 (FIGS. 1 and 2) of a packaging system such that container 240 can slide or otherwise move along tracks 140 and 150. The flange top edge 250 further comprises a third side 700 and fourth side 710. When containers 240 are arranged in a chain, side 700 of one container 240 is positioned adjacent side 710 of an adjacent container (except for the first container 240 in the chain). Top edge sides 700 and 710 of adjacent containers 240 can be connected to one another by a frangible interface 255. The containers 240 can then be detached from one another by breaking the connector 255.

Each container 240 includes protrusions or ribs 720A, 720B on the exterior of tapered body 660 of the container 240. Ribs 720A and 720B each support a tab section 730A and 730B respectively, with tabs 730A and 730B located adjacent top flange 250. The tabs 730A and 730B are configured to interact with sprocket teeth 430 (FIG. 15) on compliance wheel 410 to allow the container 240 to be moved as teeth 430 apply force to tabs 730A and 730B when wheel 410 is rotated. The ribs (720A, 720B) and tabs (730A, 730B) may in many embodiments be configured to aid in childproofing the packaging system, as described further below. Counter-clockwise rotation of wheel 410 results in teeth 430 applying force to tab 720A to move container 240 in one direction, while clockwise rotation of wheel 410 results in teeth 430 applying force to tab 720B to move container 240 in the opposite direction.

The tapered body 660 of each container 240 further comprises an arcuate or concave portion 740 positioned between the ribs 720A and 720B. Top flange 250 includes an arcuate or concave portion adjacent to the concave portion 740 of container body 660. The configuration of the concave portions 740 and 750 of the container 240 allows the teeth 430 of compliance wheel 410 to engage the tab portions 730A and 730B of the container 240 without interference from the tapered body 660 of the container 240.

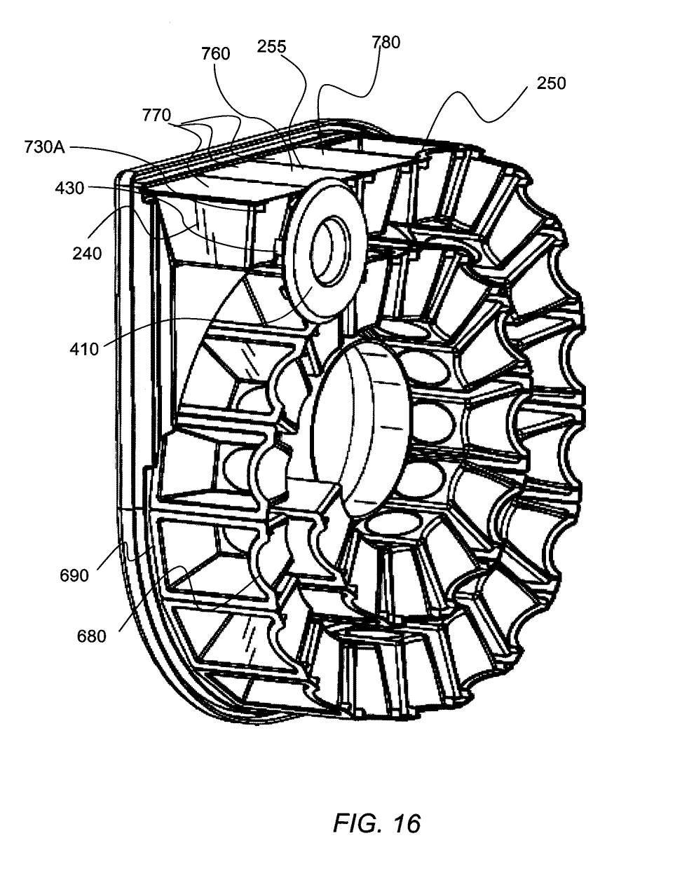

Referring now to FIG. 16, a plurality of medication containers 240 and a compliance wheel 410 are shown in association with the spiral track of shell portion 130 of a spiral packaging system (wall 120 and shell portion 110 are omitted for clarity). A flexible lidstock strip 760 provides a cover for the chain of containers 240, with the portion of lidstock strip 760 adjacent to a container providing a lid or cover 770 for that container. For clarity, lidstock strip 760 is shown only on the first three containers 240, but it should be understood that lidstock strip 760 extends to cover each of the containers in FIG. 16. The top edge sides 700 and 710 of adjacent containers 240 are joined together by the lidstock strip 760, which seals each container 240 and joins adjacent containers 240 together.

The lidstock strip 760 may be frangible or breakable at the interface or junction of sides 700, 710 to allow detachment and separation of adjacent containers 240. The lidstock strip 760 may include perforation lines (not shown at edges 700 and 710) to provide frangible interfaces 255. When thus detached, each container 240 retains a portion 770 of the lidstock strip 760 as a cover, which can be removed or broken to access the medication within the container 240.

A plurality of containers 240 may be connected to one another by a single lidstock strip 760, allowing a “chain” of containers to be easily filled, sealed and placed in the spiral groove or track of a packaging system. Each sealed container 240 may be assigned to a specific dosage period and contain the medicines required for that dosage period. The section of lidstock strip 760 adjacent to each container 240 may be printed (using thermal transfer, ink jet, laser, or other suitable electronic printing method) with patient, medicine, and dosage period information, such as patient name; D.O.B.; dosage period; date; medicine contained including type, strength, number of tablets; expiry date, and the like. The lidstock strip 760 may also be printed with images of the medicaments contained therein, and/or a photo of the patient.

Lidstock strip 760 provides a writing surface 780 where patient data, container content and prescription information can be placed. The lidstock strip 760 may be extended between adjacent sealed containers (and then folded up as it is loaded into the dispenser or packaging system) to allow for extra printable area and a larger gripping surface for removing lids 770 from containers 240. The sealed container strip or chain is designed to be filled using a tabletop automated filling system that can include automatic inspection and verification of the medication product with which containers 240 are filled.

The lidstock strip 760 is configured to attach or adhere to the flanged top surface 250 of each of the individual containers 240. Lidstock strip 760 is preferably a laminated film which is heat sealed onto the flanged tops 250 of containers 240. Lidstock strip 760 may further comprise peel tabs (not shown) which extend out past the flanged top edge 250, making the lid 770 of each container 240 removable by peeling to facilitate in the opening of the container 240. Concave portions 740 and 750 facilitate removal of lid 770 from container 240 by a user by providing a “finger groove” to allow grasping of the lid 770 (it may also include a separable plastic gripping tab and/or a feature to enable it to be connected to the dispenser body for one-handed opening). Lid 770 also comprises a printable area 780 where patient data, container content and prescription information can be placed.

The individual containers 240 are preferably manufactured from clear or tinted plastic to allow viewing of the tablets within. The individual container(s) may be made by thermoforming or injection molding techniques. Exemplary plastics utilized for the individual container comprise but are not limited to polyphenysulphone, polystyrene, polypropylene, as well as polyethylene. The lidstock strip may comprise a paper-backed plastic film or foil laminate with sealing material in the structure for a peel seal with the molded plastic sealed flanged cup body.

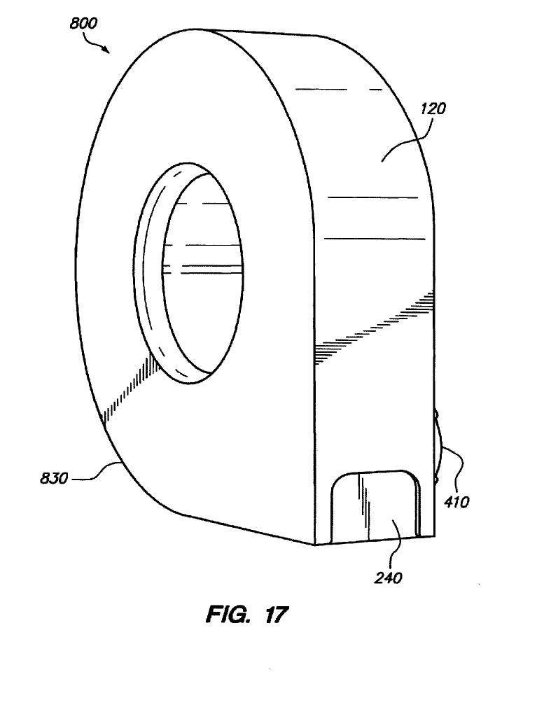

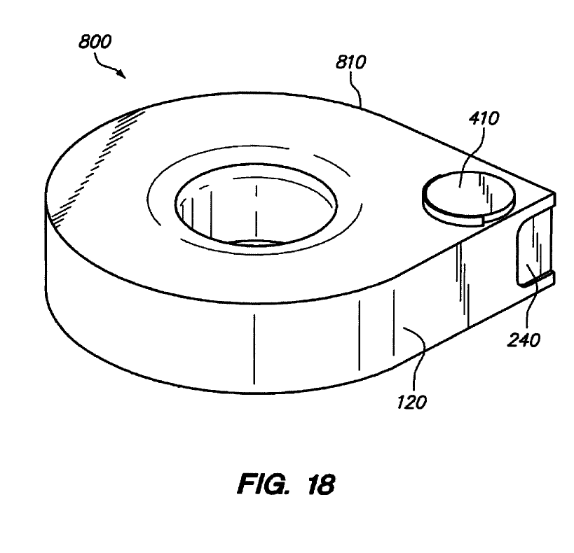

Referring now to FIGS. 17 and 18, there is shown another embodiment of a medication packaging system 800, wherein like reference numbers denote like parts. The medication packaging system 800 includes transparent outer facings or housing elements 810 and 830 that are joined to shell portions 110, and 130 as described above for the apparatus 500 in FIGS. 4-6. In the apparatus 800, the outer wall 120 is transparent. The outer wall 120 is configured to receive label strip 840 that includes a variety of printable indicia that is also referred to as “label strip” indicia. The term “label strip indicia” refers to writings, pictures and drawings that are applied to the label strip 840.

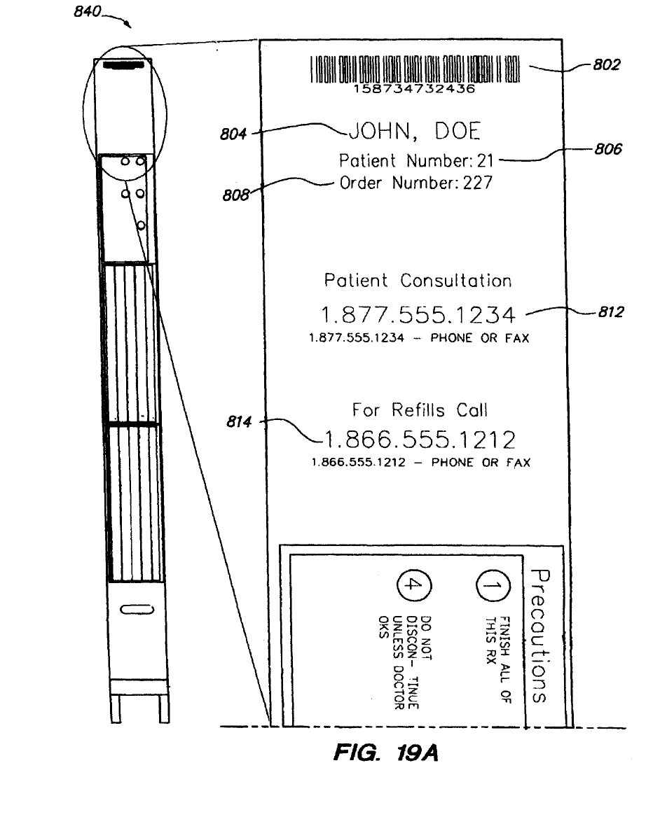

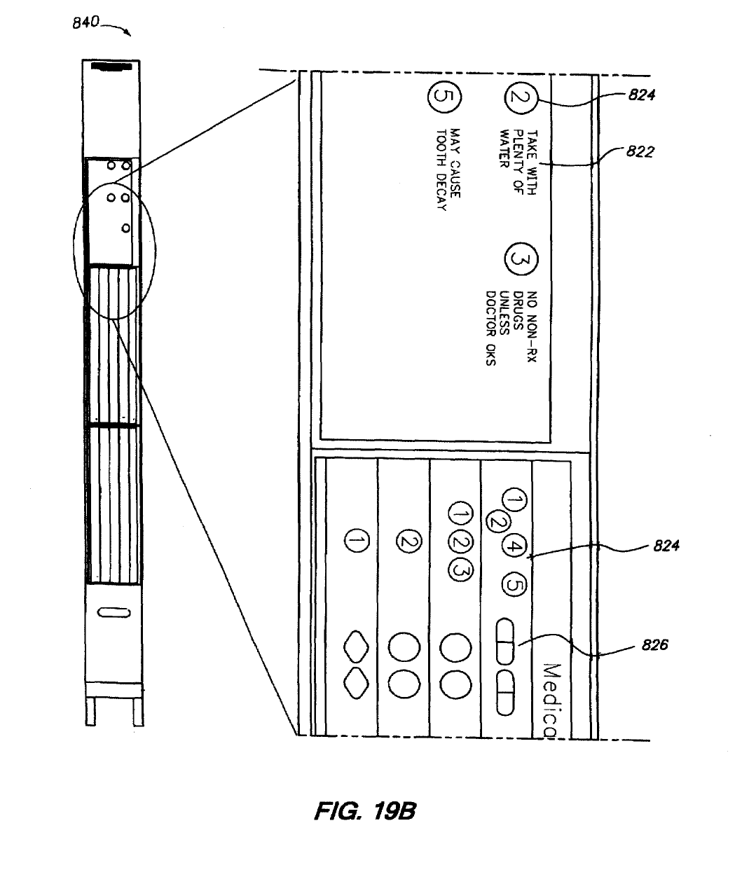

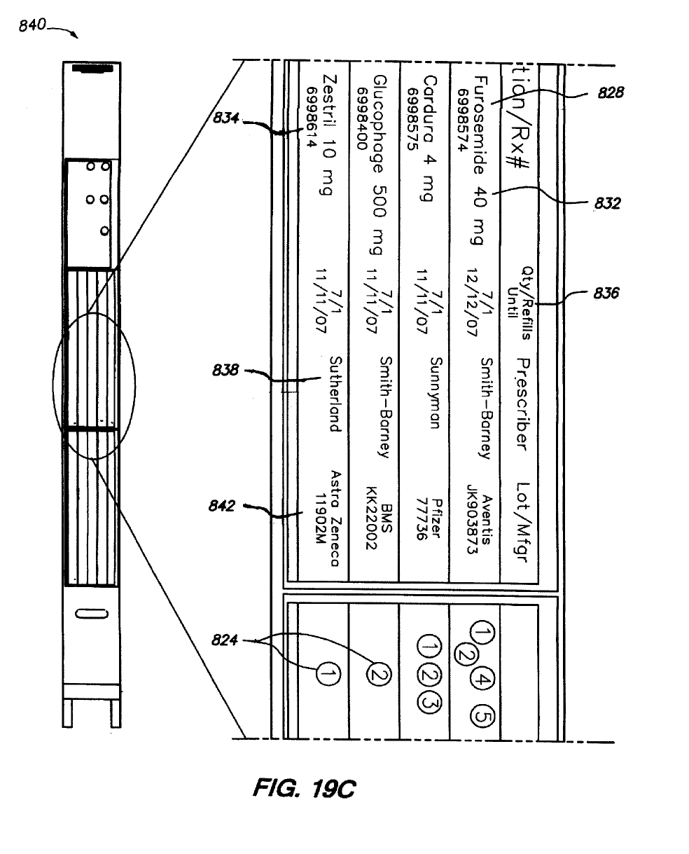

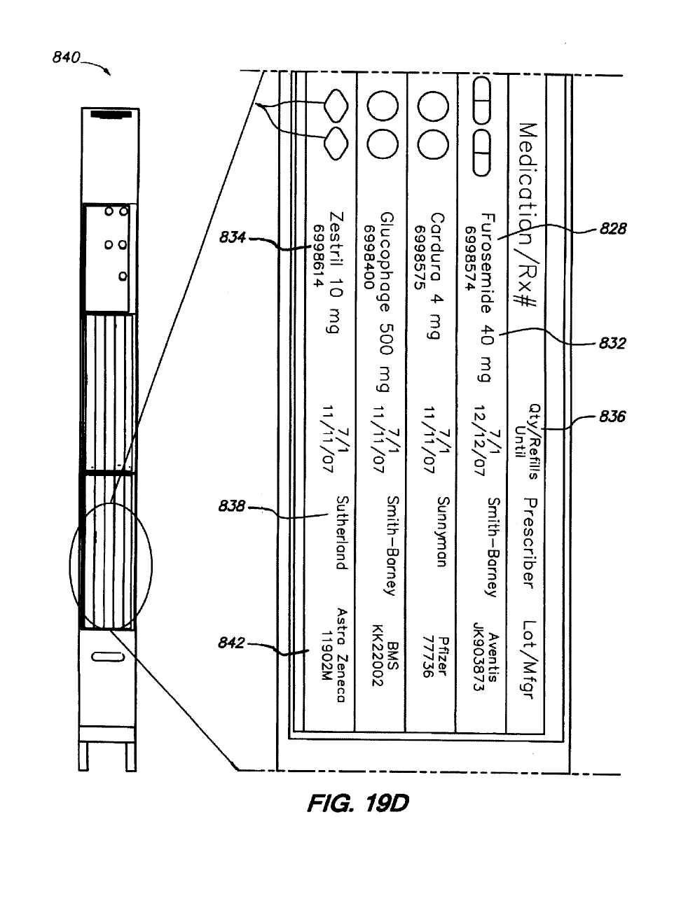

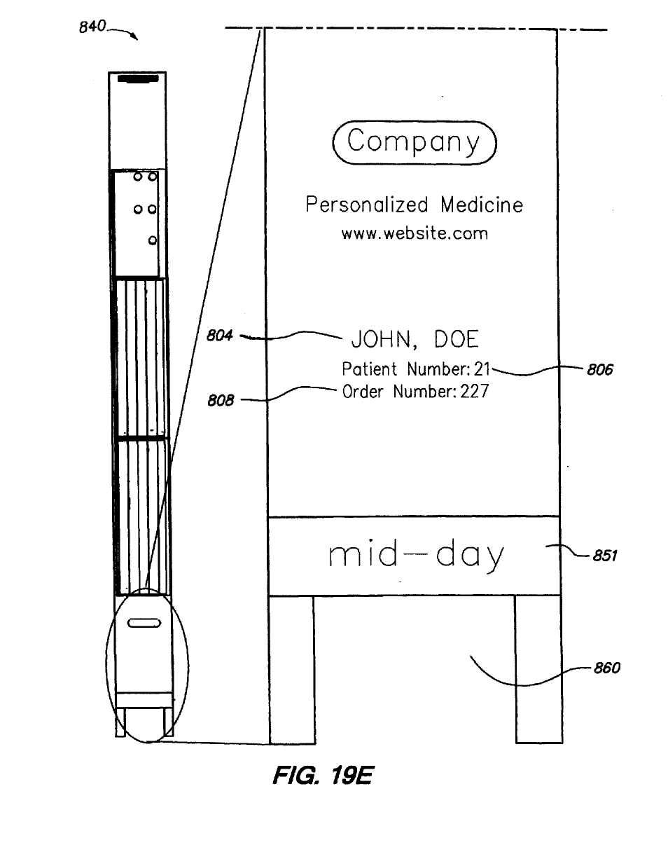

Referring also to FIGS. 19A-19E, a label strip 840 is structured and configured to fit within the apparatus 800 along the transparent outer wall 120. Label strip 840 having label strip indicia includes patient and medication data such as, for example, patient name and number, names of prescribing physicians, medicine(s), dosage strength(s), medicine quantity(s), color images of the medicine(s), prescription number(s), NDA number(s), warning(s), dosage period(s), administration schedule(s), and the like. The illustrative label strip indicia are configured such that all information necessary for drug identification and administration is affixed to the medicament container.

In an illustrative reusable embodiment, the apparatus 800 is reusable and the label strip 840 may be interchangeable and removable from the apparatus 800 when the shell portion having the outer wall is disassembled.

In an illustrative disposable embodiment, the apparatus 800 is disposable after each use, and the label strip 840 may be adhered directly to the inner or outer surface of wall 120. An opening 860 at the end of strip 840 conforms in shape to opening 230 so that opening 230 is not blocked by strip 840.

FIG. 19A shows a detailed view of a first label portion that includes a bar code 802 representing the patient order, a patient name 804, a patient number 806, an order number 808, a telephone/facsimile number 812 for patient consultations, and a telephone/facsimile number 814 for ordering prescription refills. Some embodiments, particularly those embodiments suitable for use in a long-term care facility, may also include a photograph (not shown) of the patient in this section of the label. A portion of the precautions section of the label 840 is also visible in FIG. 19A.

A detailed view of the second label section, including the remainder of the precautions section of the illustrative label 840, is shown in FIG. 19B. The drug precautions 822 are shown associated with the medicaments in the illustrative order. Each precaution is assigned a number or other easily decoded symbol for use in the next label portions. In this illustrative example, there are five precautions 822 and each is assigned to a number 824. At the bottom of the figure, the numbers 824 associated with each precaution 822 are displayed next to the medicament images 826 that depict each medication in a prescription order.

The second, third, and fourth label portions contain important information about each pharmaceutical or nutriceutical displayed in an easy-to-interpret fashion. A third label portion associated with label 840 is shown in FIG. 19C, and a fourth label portion is shown in FIG. 19D. In this illustrative embodiment, the numbers 824 associated with drug precautions 822 are listed next to the images 826 for each pharmaceutical or nutriceutical in an order (see FIG. 19B).

In FIGS. 19C and 19D, the name of the pharmaceutical or nutriceutical 828 is provided adjacent to the images 826. Note, the illustrative label displays some information more than once. Proximate to the images 826 are the corresponding dosage unit 832, the name of the pharmaceutical or nutriceutical 828, and the prescription number 834 of the pharmaceutical or nutriceutical. In the next column of text, the quantity and refill information 836 is provided, including the date the prescription expires. The adjacent column shows the prescribing physician 838 associated with each pharmaceutical or nutriceutical. The last column shows the manufacturer information 842, including lot number of each drug in the package.

A duplicate of the patient name 804, the patient number 806, and the order number 808 are included in the last portion of the label, shown in FIG. 19E. Furthermore, the second label portion 843 includes a field for the period of day or time interval 851 during which the pharmaceuticals or nutriceuticals are consumed. In the illustrative time interval 851, the time period is “mid-day.”

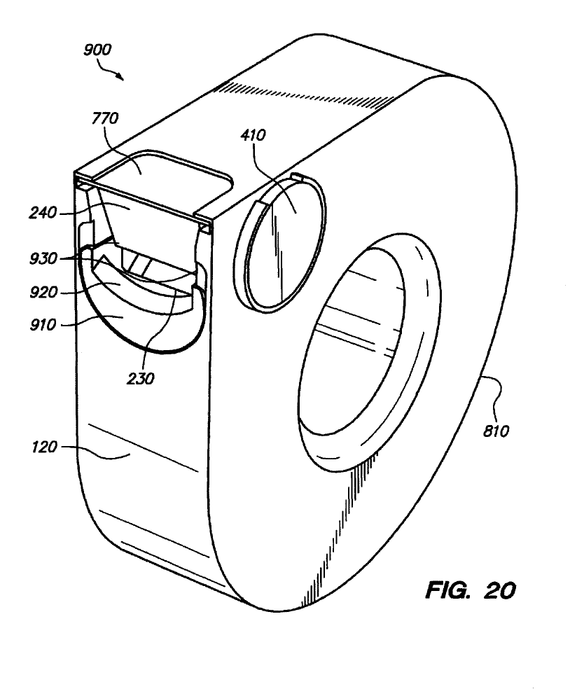

Referring now to FIG. 20, there is shown still another embodiment of a medication packaging system 900, wherein like reference numbers are used to denote like parts. The apparatus 900 includes a child proofing barrier element 910 movably mounted in wall 120 adjacent to opening 230. Barrier 910 is movable between an open position (as shown in FIG. 20) that allows container 240 to pass through opening 230, and a closed position (not shown) wherein container 240 is blocked by barrier 910 such that container 240 cannot exit or be removed through opening 230. A spring or bias element (not shown) may be operatively coupled to barrier 910 such that barrier 910 remains in the closed position unless a user applies sufficient force to barrier 910 to overcome the bias and move barrier 910 to the open position.

In the embodiment of FIG. 20, barrier 910 includes an arcuate or curved lip 920 that is configured to cover and block corners 930 of container 240 when barrier 910 is in the closed position, and which allows corners to pass over or clear lip 920 when barrier is in the open position as shown in FIG. 20.

To operate the apparatus 900, a user simultaneously pushes on or depresses barrier 910 to the open position while advancing compliance wheel to move container 240 through opening 230. When the container 240 has passed through opening 230, the container 240 is detached and removed from the apparatus 900, and the barrier 910 is released to return to the closed position. The next container 240 is then blocked by barrier 910 from exiting the apparatus 900 until the child proofing barrier 910 is again depressed or moved to the open position. Simultaneous manipulation of the compliance wheel 410 and barrier 910 are sufficiently difficult that small children are unable to access the medication within apparatus 900.

The medication packaging systems may be modular and attachable to each other to form extensible or scalable medication packaging systems. FIG. 21 shows two medication packaging systems 900A and 900B joined together to form an extended medication packaging system 940. In using the apparatus 940, a patient accesses medication containers 240 in packaging system 900A by depressing child proofing barrier 910, rotating compliance wheel 410, and detaching and removing the container 240 as described above. When all medication containers 240 have been dispensed or removed from the packaging system 900A, it is detached from the packaging system 900B. The compliance wheel on packaging system 900B is then exposed, and the user may then access the medication therein in the same manner. In this embodiment the individual packaging systems 900A and 900B are modular and additional packaging systems (not shown) may be attached to the extended system 940.

Alternatively, systems 900A and 900B may comprise a “morning” or “AM” module and an “evening” or “PM” module that are joined together for convenience but which are easily detachable and re-attachable. A user would, for example, access the AM medication from system 900A, then later would detach the AM medication system 900A to access the PM medication from system 900B. The two modular systems 900A and 900B are then re-attached together for convenient storage until the next use.

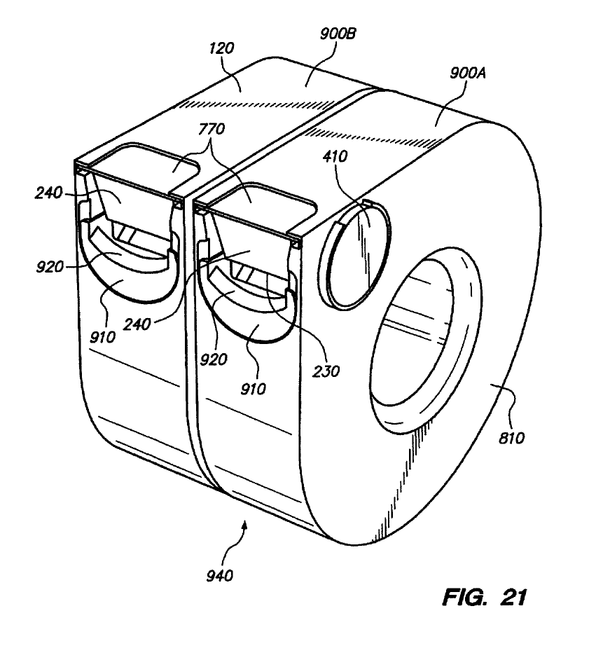

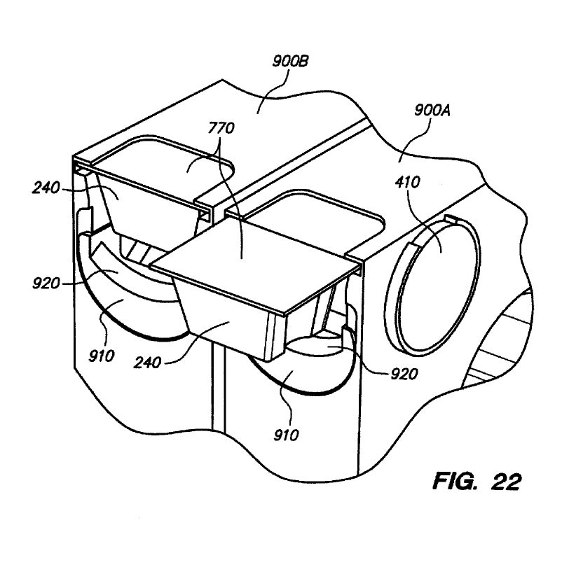

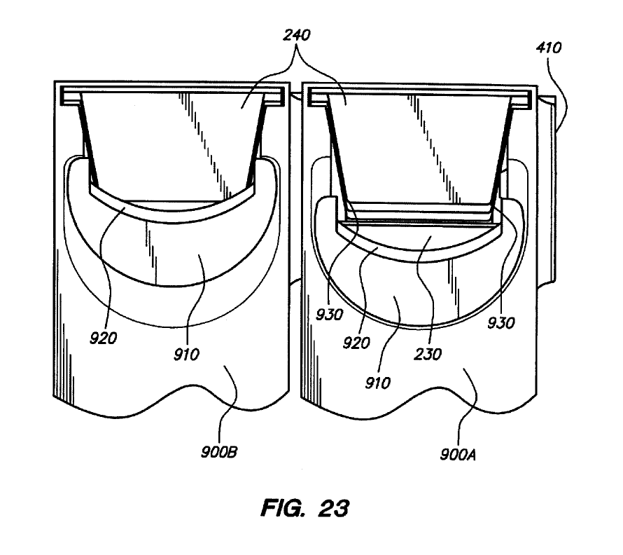

Operation of the child proof barrier 910 will be more fully understood by making reference to FIGS. 22 and 23, wherein like reference numbers denote like parts. FIGS. 22 and 23 show a portion of the modular system 940 wherein the child proof barrier 910 in packaging system 900B is shown in the closed position, and wherein the barrier 910 is shown in the open position in adjacent packaging system 900A. In the closed position as shown in system 900B, arcuate lip 920 of barrier 910 blocks the lower corners of container 240 to prevent container 240 from being removed through opening 230. In the open position shown in system 900A, the lower corners 930 of container 240 are clear of lip 920 and container 240 may be advanced through opening 230 by operation of compliance wheel 410. The arcuate shape of lip 920 accommodates a user's finger and facilitates manipulation of barrier 910 and container 240.

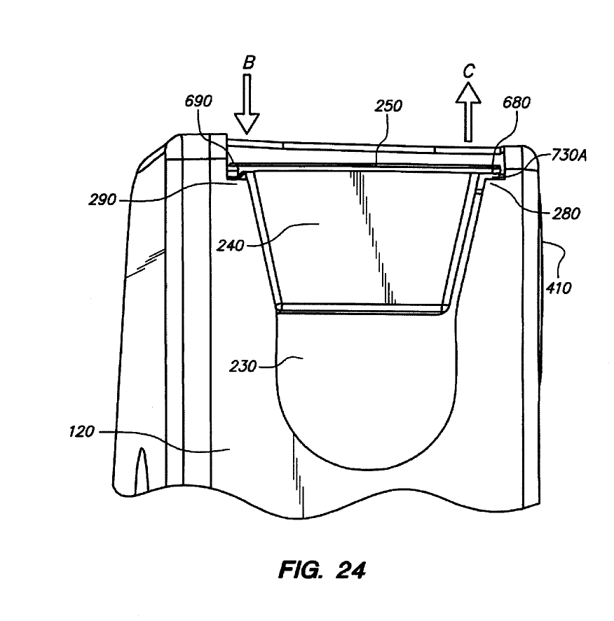

Referring now to FIG. 24, a portion of the medication packaging system 500 of FIGS. 9-11 is shown with a child proof mechanism to prevent unwanted access by children to the medication contained in the apparatus 500. In FIG. 10, aperture 230 includes indents 280 and 290 that allow corresponding portions 680 and 690 of the flange 250 on container 240 to pass through opening 230 as compliance wheel 410 is rotated. In the embodiment of FIG. 24, indent 290 is slightly lower or offset with respect to indent 280, so that flange edge 690 can pass over indent 290, but flange edge 680 cannot pass over indent 280 and is blocked by wall 120 adjacent indent 280. When container 240 and/or flange 250 are made of a resilient or flexible material, applying a downward force on flange portion 690 as shown by arrow B results in an upward movement of flange portion 680 as container 240 and flange 250 flex in response to the force indicated by arrow B. The upward movement of flange portion 680 allows it to clear wall 120 past indent 280 so that container 240 may be removed from opening 230. Thus, a user simultaneously applies a downward force against flange portion 690 while rotating compliance wheel 410 to access the medication within packaging system 500. Simultaneous manipulation of flange edge 690 and compliance wheel is too difficult for young children to achieve, and the packaging system 500 is effectively child-proofed.

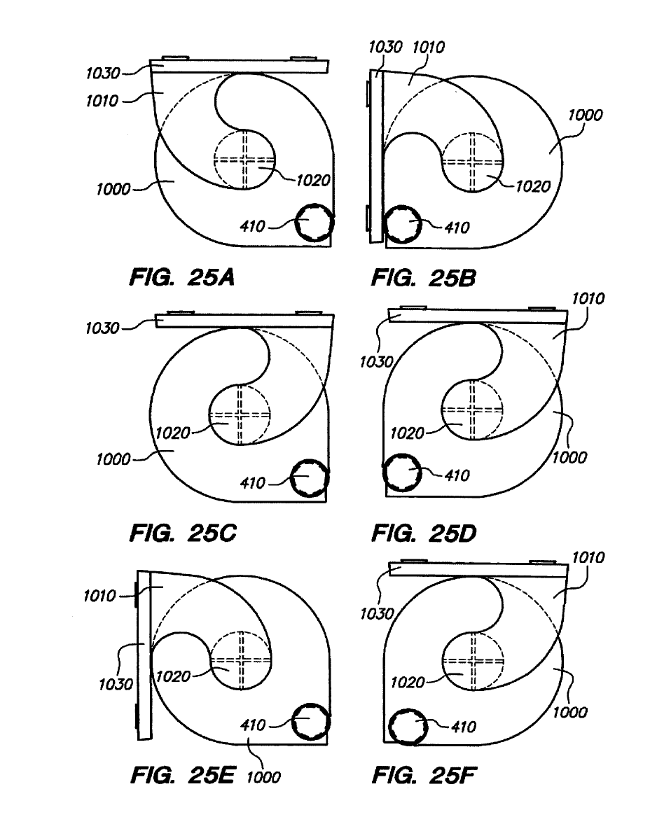

Referring now to FIG. 25, a medication packaging system 1000 in accordance with the invention is shown mounted onto a support arm 1010, with like reference numbers denoting like parts. The central opening in the packing system 1000 provided by the openings in the shell components and housing elements as described above allow system 1000 to be rotatably mounted on a disc 1020 on support arm 1010. Support arm 1010 in turn is coupled to a mounting bracket 1030 which may be fastened to a vertical or horizontal surface. The medication packaging system 1000 thus may be conveniently mounted on the bottom of a medicine cabinet, on a counter top, or other surface, in a variety of orientations selectable by a user.

It is to be understood that the foregoing is a detailed description of illustrative embodiments. The scope of the claims is not limited to these specific embodiments or examples. Therefore, various elements, details, execution of any methods, and uses can differ from those just described, or be expanded on or implemented using technologies not yet commercially viable, and yet still be within the inventive concepts of the present disclosure. The scope of the invention is determined by the following claims and their legal equivalents.

What is claimed is:

1. A child proof medication packaging system comprising:
a dispenser having a plurality of medicament containers housing a plurality of different medications, the dispenser including an aperture configured to allow the medicament containers to exit the dispenser through the aperture; and
a child safety component associated with the aperture in the dispenser, the child safety component adjustable to a first position and a second position and configured to prevent the medicament containers from passing through the aperture when in the first position, and to permit the medicament containers to pass through the aperture when in the second position.

2. The child proof medication packaging system of claim 1 wherein the child safety component includes a slidable barrier mounted in the dispenser adjacent the aperture, the barrier slidably movable from a closed position wherein the barrier prevents the medicament containers from passing through the aperture, and an open position wherein the medicament containers can pass through the aperture.

3. The child proof medication packaging system of claim 1 wherein the dispenser comprises a first shell and a second opposing shell and a wall portion connecting the shells and the aperture is included in the wall.

4. The child proof medication packaging system of claim 3 wherein the dispenser further comprises a spiral track on at least one of the shells, the plurality of medicament containers movable along the track.

5. The child proof medication packaging system of claim 4 wherein each of the medicament containers comprises a flanged top edge that engages the spiral track.

6. The child proof medication packaging system of claim 4 further comprising a first housing portion and a second housing portion mounted respectively on the first shell and the second shell.

7. A child proof medication packaging system, comprising:
a dispenser having a plurality of medicament containers housing a plurality of different medications, the dispenser including an aperture configured to allow the medicament containers to exit the dispenser through the aperture; and
a security means for preventing access to the medicament containers in the dispenser, the security means associated with the dispenser and adjustable to a first position and a second position, the security means configured to prevent the medicament containers from passing through the aperture when in the first position, and to permit the medicament containers to pass through the aperture when in the second position.

8. The patient compliant medication management system of claim 7 wherein the security means comprises a child safety component associated with the aperture in the dispenser, the child safety component adjustable to a first position and a second position and configured to prevent the medicament containers from passing through the aperture when in the first position, and to permit the medicament containers to pass through the aperture when in the second position.

9. The patient compliant medication management system of claim 7 wherein the security means comprises a child safety component that includes a slidable barrier mounted in the dispenser adjacent the aperture, the barrier slidably movable from a closed position wherein the barrier prevents the medicament containers from passing through the aperture, and an open position wherein the medicament containers can pass through the aperture.

10. The child proof medication packaging system of claim 7 wherein the dispenser comprises a first shell and a second opposing shell and a wall portion connecting the shells and the aperture is included in the wall.

11. The child proof medication packaging system of claim 10 wherein the dispenser further comprises a spiral track on at least one of the shells, the plurality of medicament containers movable along the track.

12. The child proof medication packaging system of claim 11 wherein each of the medicament containers comprises a flanged top edge that engages the spiral track.

13. The child proof medication packaging system of claim 11 further comprising a first housing portion and a second housing portion mounted respectively on the first shell and the second shell.

14. A method for dispensing medication, comprising:
housing a plurality of medicament containers in a dispenser, in which each medicament container includes medication associated with a dosage regimen, the dispenser including an aperture configured to allow the medicament containers to exit the dispenser through the aperture, the dispenser including a child safety component associated with the aperture that is adjustable to a first position and to a second position,
preventing the medicament containers from passing through the aperture when in the child safety component is in a first position; and
permitting the medicament containers to pass through the aperture when in the second position.

15. The method of claim 14 further comprising:
adjusting the child safety component to the second position;
advancing the medicament containers towards the aperture such that one of the medicament containers passes through the aperture; and
returning the child safety component to the first position.

16. The method of claim 15 further comprising detaching one of the medicament containers that has passed through the aperture.

17. The method of claim 16 wherein the detaching one of the medicament containers that has passed through the aperture from the medicament carriers further comprises breaking a frangible interface connecting the medicament container that has passed through the opening with the medicament carriers.

18. The method of claim 17 wherein the advancing the medicament containers towards the aperture such that one of the medicament containers passes through the aperture further comprises sliding the medicament containers along a spiral track.

19. The method of claim 18 wherein each of the medicament containers comprises a flanged top edge that is configured to engage the spiral track.

20. The method of claim 19 wherein the dispenser further comprises verifying that one of the medicament containers has been detached from the medicament containers with compliance indicia on the dispenser that is associated with the corresponding medicament container that has been detached.
